(19) United States
(12) Patent Application Publication
(10) Pub. No.: US 2008/0161778 A1
(43) Pub. Date: Jul. 3, 2008

(54) Fluid isolation device and method for treatment of fistulas

(76) Inventor: Brent Steward, (US)

Correspondence Address: BRUCE J. CLARK, P.O. BOX 3555, TOPEKA, KS, 66601, US

(21) Appl. No.: 11/649,147

(22) Filed: Jan. 2, 2007

(51) Int. Cl. A61M 27/00

(52) U.S. Cl. 604/543

(57) ABSTRACT

A fluid isolation device for allowing collection and control of fluids from fistulas and other openings has a rigid flange, a tubular passageway having two ends, balloon fluid filled sealing means. In one mode, it is used with porous foam and occlusive adhesive dressing to allow the creation of two pressure zones such that the fluids can be collected and controlled through a neutral pressure zone isolated from a surrounding negative pressure zone.

### BACKGROUND OF THE INVENTION

The invention pertains to the field of control of open wounds, fistulas and other internal openings, including those where the presence of undesirable body fluids coming from the fistula interfere with the healing of the wound and the fistula itself or where an otherwise difficult seal is necessary to remove often undesirable body fluids.

Open wounds of the body remain a difficult treatment problem. In the presence of such a wound, the natural protective barrier of the skin cannot be maintained, and thus the problem often represents a life-threatening situation. Strategies for treatment include acceleration of natural closure, primary surgical closure, or temporization. The temporization strategy allows either optimization of the patient's condition (which in turn can allow a direct closure), or allows maintenance while natural closure occurs. Standard ostomy appliances are often limited in ability to control these fluids, due to an inability to seal to surrounding tissues.

One particularly difficult and life-threatening instance of open wounds exists when the wound contains a draining fistula. This represents multiple threats. Fluid output can be caustic or damaging to surrounding tissues both within and surrounding the wound. Fluid and electrolyte losses are often dangerous and potentially life-threatening. In the case of the enterocutaneous fistula, nutritional and immune status can be impossible to maintain appropriately with the gut rendered unusable.

Collection and control of fistula output can be problematic in terms of attachment of any type of collection device, particularly in the instance of an open abdominal wound. The irregular geometry does not allow standard sealing techniques and are prone to leakage. The open wounds' surface is often moist and slime-coated, which prevents adhesives from appropriate attachment. The viscera are also extremely fragile. They can become easily damaged by attempts at closure, manipulation, or attachment of collection devices.

The strategies of natural healing, primary surgical closure, or temporization all have limitations, and these are very acute in the setting of a fistula. Natural healing is an extremely slow process, and with poor ability to stabilize the patient's condition for this long period of time, will often fail. Primary surgical closure can be problematic because of the patient's inability to tolerate surgical intervention, the high rate of complications associated with this, and with outright inability to accomplish the desired closure. Temporization is often the preferred choice in these cases, but often the patient cannot be maintained in the longer time frame, either in terms of local wound care/control or in terms of the patient's overall condition.

Negative pressure wound therapy, or vacuum assisted closure, (VAC) advanced the state of the art to treat wounds. This therapy allows a negative pressure zone to be applied to the entire wound surface. This is combined with a foam dressing which facilitates granulation and healing of the wound bed. This maintains a healthy wound environment and controls fluid output.

A relative contraindication to VAC therapy is, however, the presence of a fistula. There are multiple reasons for this. The negative pressure may increase fistula output and make the fistula larger. The VAC system quickly becomes fouled by the fistula output and fails.

The apparatus and method disclosed is designed to overcome these obstacles. It provides an area of vacuum isolation or exclusion within the negative pressure environment. The device is held in place by the negative pressure VAC dressing, and allows a stable and protected interface for attachment of various drainage collection devices. With the device in place, the negative pressure wound therapy can be continued, the fistula output can be controlled, and the patient's status maintained. With a reliable method for controlling enteric output, the gut can be rendered usable for enteric feeding. All of the above allow more effective temporization for either natural closure or more optimal primary surgical closure.

It is thus an object of the invention to provide an apparatus and method to create an effective seal around a fistula so as to allow collection of fluids leaking.

It is a further object of the invention to provide an apparatus and method for creating such a seal in combination with vacuum assisted closure technology. Other objects and features of the invention will be apparent from the specification herein.

### DESCRIPTION OF THE DRAWINGS

FIG. 1 is a front view of the device in place, showing the negative and neutral pressure zones in and around the body area.

FIG. 2 is a front perspective view of the invention showing adjustable means.

FIG. 3 is an isolated front perspective view of the invention.

FIG. 4 is a top perspective view of the invention including the surrounding foam.

FIG. 5 is a front view of an alternative mode of the invention.

FIG. 6 is a front view of an alternative mode of the invention.

### DESCRIPTIONS OF THE PREFERRED EMBODIMENTS

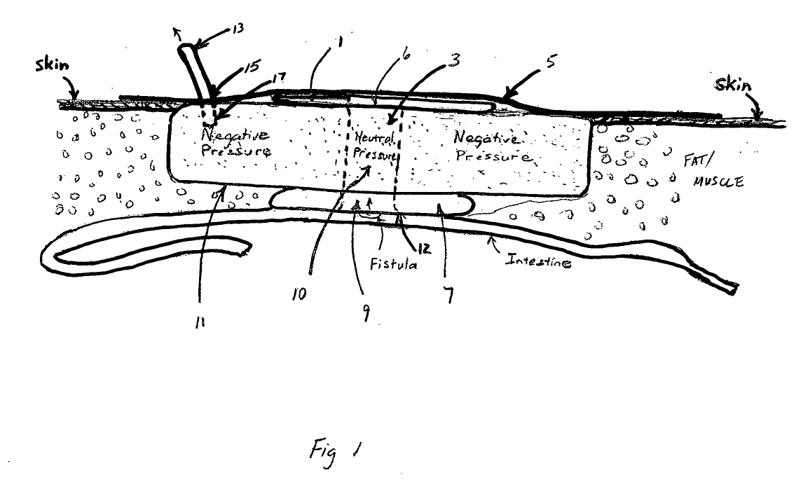

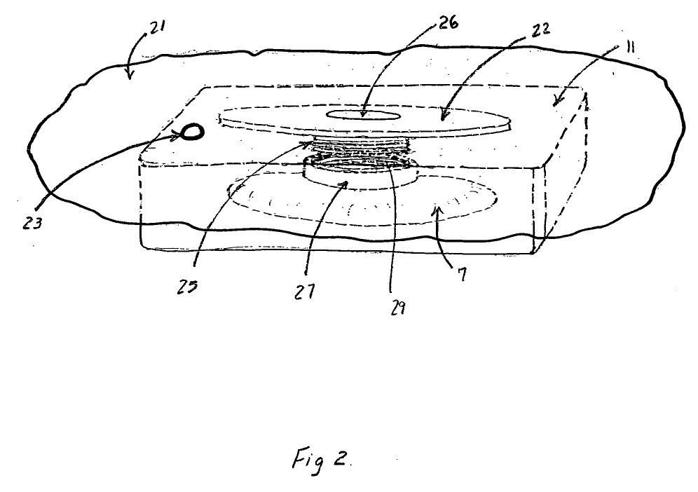

The invention is shown in place in FIG. 1. Rigid flange 1 has an opening 6 that is adjacent a preferably rigid tubular passageway 3, which passageway extends to the balloon type toroidal sealing means 7, which sealing means has opening 9. The sealing means opening is placed to fully encompass the fistula. The rigid flange 1, the tubular passageway 3, and the sealing means 7, combine to form a sealed passageway 10 that is able to maintain at local atmospheric pressure a neutral pressure zone, while maintaining a surrounding negative pressure zone (less than atmospheric pressure) sufficient to create and maintain the seal around the opening of the toroidal balloon means at 12. Occlusive dressing 5 having adhesive is placed over the flange and across the skin to create the entire seal for the VAC. The negative vacuum, created using a vacuum device attached to tube 13 entering through the dressing at 15, also draws the toroidal balloon sealing means tight over the area around the fistula at 12. Collection devices are attached at the opening 6 by cutting the desired hole through to the rigid flange opening 26 (FIG. 2).

The isolated invention is shown in FIG. 2 in perspective having means to adjust the extension of the device so as to fit the desired area in the body. The flange, tubular passageway and the toroidal sealing means comprise the sealing/passageway assembly, but in FIG. 2 it is comprised of two sections. The first section is comprised of the flange means 22, and a tubular section attached to it having male (or female, as desired) threaded means. The second section is comprised of toroidal balloon sealing means 7 attached to a (preferably rigid) tubular member which itself has corresponding and opposite female (or male) threaded means. These two sections may thus be connected and adjusted during placement to fit the application.

The adhesive dressing 21 is shown overlaid, having vacuum access means 23.

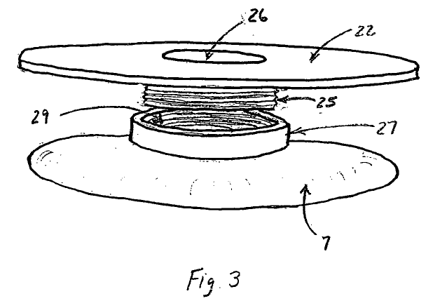

FIG. 3 shows another view of the adjustable version of the invention. Other adjustment means are envisioned.

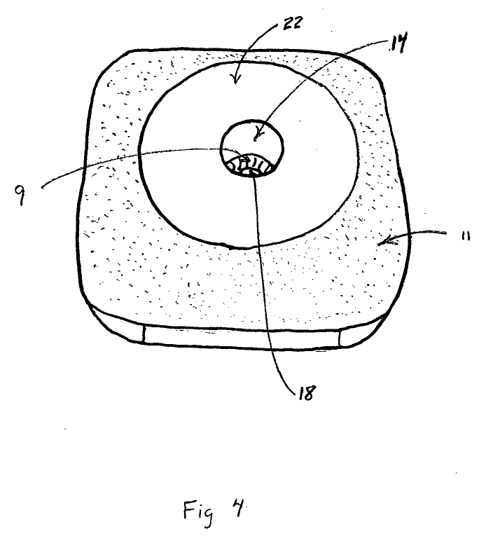

FIG. 4 shows a top perspective, with the foam in place surrounding the device, and showing the tubular means 14, preferably rigid, creating the passageway. Tubular here refers to any nonporous material and shape that has a passageway therein and maintains the difference between the two pressure zones in FIG. 1. Toroidal balloon means 9 has opening 18.

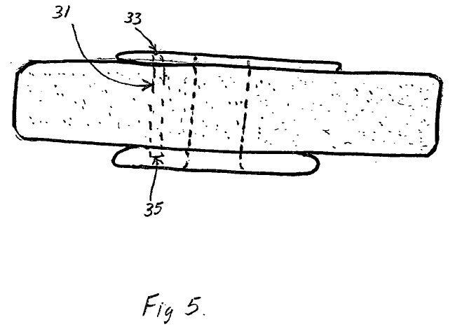

FIG. 5 shows an alternative of the invention with means for adjusting the pressure in the toroidal balloon. Air or other fluid can be pumped in using tube means 31 at opening 33 extending to the balloon means at 35.

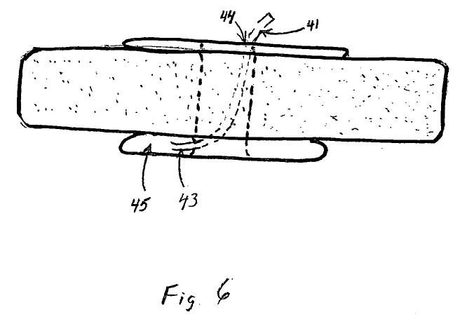

FIG. 6 shows a still alternative means, with the tube 41 simply extending through the passageway, a neutral pressure area.

It should be understood that the toroidal balloon sealing means can be various nontoroidal shapes. ‘Balloon’, as used here, includes any fluid (including liquid or air) filled flexible device sufficient to provide sealing means, or any flexible non fluid filled device that is nonporous sufficient to provide sealing means, altogether defining compressible sealing means.

The method, and manner of use, is as follows.

VAC technology suggests the use of a foam and occlusive adhesive dressing.

The foam dressing is selected for use on the entire open wound and fitted as desired. A hole or defect is created in the foam so as to allow the tubular means to fit therein. The tubular means is fitted by adjusting the tube length, screwing or unscrewing as desired (or a proper size is selected from a variety of sizes if it is not adjustable) such that the tubular length is approximately the thickness of the foam dressing. This length may need to be adjusted later. The pressure of the balloon on the tissue interface increases with the lengthening of the tube and adjustment of this may be needed either to create a better seal or to prevent excessive pressure at the tissue interface.

The assembly is placed on the wound area such that the fistula is contained within the neutral pressure zone. The bottom surface of the balloon means should be in complete contact with the tissue surrounding the fistula and the foam should be in contact with the remainder of the open wound.

The occlusive adhesive dressing is applied over the wound, the surrounding tissue, and the entire assembly, assuring complete contact with the flange area and surrounding tissue so as to provide a continuous seal extending circumferentially from the flange to the tissue surrounding the wound area. This creates two zones in relation to atmospheric pressure, one negative and one neutral, where the neutral zone is within the tube and contains the fistula and the negative pressure zone is applied to the remainder of the wound.

The vacuum is created in the negative pressure zone by standard VAC techniques. The vacuum tube is attached through the dressing to the foam, and the vacuum pump is then attached to create the vacuum in the negative pressure zone.

Create an opening in the dressing at the flange to access the neutral pressure zone.

Check to assure the desired negative pressure is maintained in the negative pressure zone. The foam dressing should remain compressed by the negative pressure. If this is not maintained all interfaces between neutral pressure zones should be checked for leakage. These should include all interfaces of the occlusive dressing (both with the surrounding tissue and the flange) and the interface of the balloon means with the tissue. Means of resolving leaks at the balloon means include adjustment of the fill volume within the toroidal balloon and adjustment of the tube length.

Once the seals are assured, and the negative pressure zone maintained, a desired collection device may be attached to the flange opening.

Thus what is shown is a device and method that provides a relatively inexpensive, easy to use and reliable technique for treatment of fistulas in often life threatening and complex situations created by the presence of uncontrolled toxic, caustic, or other undesirable fluids. The device and method allows for control and isolation of these fluids.

I claim:

1. A device for controlling fluid drainage from a fistula or other wound, comprised of:
a) a flange having an outer surface serving as a contact surface for receiving a dressing used to hold the flange in place, said flange having an opening therein.
b) tubular means having a passageway therein and having first and second end openings, said first end opening attached to the flange opening;
c) flexible sealing means attached to the second end, and having an interior passageway therethrough continuous with the tubular means passageway

2. The device in claim 1 and having:
a. Porous foam surrounding the tubular means.
b. Adhesive occlusive dressing attachable across the surface sufficient to create a seal.

3. The device in claim 1 or 2 having means for adjusting the length of the tubular means.

4. The device in claim 1 where the tubular means is comprised of two threaded members, one male, one female, each for receiving the other to allow length adjustment of the tube.

5. The device in claim 1, 2, 3, or 4 where the sealing means is comprised of compressible means.

6. The device in claim 5 wherein the compressible means are adjustable to create various compression rigidity.

7. A process for controlling fluid drainage from a fistula using an isolation device that provides for sealing to surrounding skin or tissues, said device having a top flange member, said flange having a top end, a tubular member attached to the flange opposite the top end, compression sealing means attached to the tubular member and having interior opening, all so as to create an interior sealed passageway through all members, and utilizing a dressing, the process comprised of the steps of:
a. Applying the device to isolate the fistula inside the interior passageway of the device
b. Create a seal of the compression means to the tissue surrounding the fistula,
c. Apply the dressing across the top of the flange extending to at least the adjacent skin, to keep the device in place with the seal.

8. The process in claim 7 utilizing porous foam, occlusive adhesive dressing, and negative pressure means applied to the foam, comprised of:
a. Selecting and fitting the desired foam dressing appropriate for the wound and fistula.
b. Creating a hole so as to allow the tubular means to fit through the foam at the appropriate location for the fistula.
c. Fitting the device so as to have a tubular length appropriate for the foam.
d. Placing the device on the wound area so that the fistula is contained within the passageway and the foam is contacting primarily the areas outside the device
e. Creating a seal between the compression means and the tissue essentially surrounding the fistula.
f. Applying the occlusive adhesive dressing over the flange and foam and extending to the skin circumferentially.
g. Creating a negative pressure area essentially contained under the occlusive adhesive dressing, excluding the passageway, utilizing the negative pressure means, and so as to maintain an essentially neutral pressure area within the passageway.
h. Create an opening in the dressing at the flange to access the neutral pressure zone.

9. The process in claims 7 or 8 having the additional step of attaching a desired fluid control device to the flange
